(19) United States
(12) Patent Application Publication
(10) Pub. No.: US 2012/0070518 A1
(43) Pub. Date: Mar. 22, 2012

(54) METHODS AND COMPOSITIONS FOR TREATING PSORIASIS

(75) Inventors: Longgui Wang, Flushing, NY (US); Xiao Mei Liu, Flushing, NY (US); Simon K. Mencher, New York, NY (US); James P. McCarron, JR., New York, NY (US)

(73) Assignee: NATROGEN THERAPEUTICS INTERNATIONAL, INC., New York, NY (US)

(21) Appl. No.: 13/239,230

(22) Filed: Sep. 21, 2011

Related U.S. Application Data
(63) Continuation-in-part of application No. 12/972,908, filed on Dec. 20, 2010.
Application No. 12/972,908 is a continuation-in-part of application No. 11/494,362, filed on Jul. 26, 2006, now Pat. No. 7,855,223.
Application No. 11/494,362 is a continuation-in-part of application No. 10/754,547, filed on Jan. 12, 2004, now abandoned.
(60) Provisional application No. 61/385,461, filed on Sep. 22, 2010.

(51) Int. Cl. A61K 36/886; A61K 36/00; A61P 17/06; A61K 31/404

(52) U.S. Cl. 424/744; 514/414; 424/725

(57) ABSTRACT

The present invention is directed to topical compositions and methods for topically treating psoriasis comprising meisoindigo as an active ingredient. The topical compositions preferably further comprise skin penetration enhancers, pharmaceutical surfactants and solubility enhancers, oil phase components, aqueous phase components, emulsifiers, moisturizers, antioxidants, vitamins, lubricants, herbal extracts, preservatives, and other ingredients. In addition to meisoindigo, the compositions may comprise other agents commonly used to topically treat psoriasis. The disclosed methods have been demonstrated to effectually treat moderate and severe forms of psoriasis to a degree similar to a potent corticosteroid without side effects. Preferably, the method involves topical application of the disclosed compositions to affected epidermal areas twice a day. In addition, the disclosed methods may be used in conjunction with other common therapies in the treatment of psoriasis.

### CROSS-REFERENCE TO RELATED APPLICATIONS

The present application is related to and claims the benefit of U.S. Provisional Application No. 61/385,461, filed Sep. 22, 2010, and is a continuation-in-part of U.S. patent application Ser. No. 12/972,908, filed Dec. 20, 2010, which is a continuation-in-part of U.S. patent application Ser. No. 11/494,362, filed Jul. 26, 2006, now U.S. Pat. No. 7,855,223, which is a continuation-in-part of U.S. patent application Ser. No. 10/754,547, filed Jan. 12, 2004, abandoned, the contents of each of which are herein incorporated by reference for all purposes.

### FIELD OF THE INVENTION

The present invention is directed to topical compositions and methods for treating psoriasis comprising meisoindigo as an active ingredient.

### BACKGROUND OF THE INVENTION

Psoriasis is a chronic, non-contagious skin disorder that appears in many different forms and can affect any part of the body. The most common type of psoriasis is plaque psoriasis, occurring in 80% of people suffering from the disease. Plaque psoriasis is characterized by red patches and lesions that are covered by a build up of skin cells that are often called scales, and most commonly seen on the elbows, knees, scalp and back. Psoriasis is classified as mild, moderate, or severe, depending on the percentage of body surface involved and severity of the disease. According to the National Institutes of Health, psoriasis is one of the most common human skin disorders, affecting greater than 3% of the United States population, or more than 5 million adults, of which greater than 1.5 million are considered to have a moderate to severe form of the disease. Although psoriasis is not fatal, it negatively impacts quality of life to a degree similar to heart disease and arthritis (Rapp et al. 1999). In addition, 10-30% of patients with psoriasis also develop a form of arthritis—psoriatic arthritis, which damages the bone and connective tissue around the joints. Furthermore, inflammatory mediators associated with psoriasis may increase the risk of obesity, diabetes, thrombosis, and atherosclerosis (Davidovici et al. 2010).

Psoriatic scales are a result of a hyperproliferative epidermis with premature maturation of keratinocytes and incomplete cornification. The mitotic rate of the basal keratinocytes is increased compared with that in normal skin, resulting in a thickened epidermis (Nestle et al. 2009). Specifically, psoriatic lesions likely evolve as interplay between cells and mediators of the immune system. Initial triggers such as physical trauma or bacterial products start a cascade of events, including the production of TNF-\acute{α}, interferon-\acute{α}, interferon-γ, IL-1β, and IL-6 by innate immune cells, which leads the activation of myeloid dendretic cells. These activated dendretic cells migrate into draining lymph nodes, present antigens to T cells, and secrete mediators, for example IL-12 and IL-23, causing the differentiation of naïve CD4+ lymphocytes into effector T cells, such as type 17 and type 1 helper T cells (Th17 and Th1, respectively). In turn, these effector cells recirculate and slow down in skin capillaries in the presence of selectin-guided and integrin-guided receptor-ligand interactions (Nestle et al. 2009). Subsequently, effector T cells secrete proinflammatory mediators such as IL-17A, IL-17F, IL-22, interferon-γ, and TNF-\acute{α}, that activate keratinocytes, leading to the production of antimicrobial peptides, inflammatory cytokines, chemokines, and 5100 proteins, which feed back into the disease cycle and shape the proinflammatory infiltrate (Nestle et al. 2009).

Although there is currently no cure for psoriasis, the disease can be treated with compounds that topically or systemically inhibit inflammation, cell proliferation, or cell differentiation. (Ehrlich et al. 2004). Candidates for topical therapies usually have plaque psoriasis affecting less than 5% of their body surface area (BSA). Various topical and systemic therapies include anti-inflammatory agents (e.g., glucocorticoids), analgesics, chemically synthesized disease-modifying antirheumatic drugs (DMARDs; e.g., methotrexate and ciclosporin), antiproliferative agents (e.g., retinoids and vitamin D analogs), TNF-\acute{α} blockers (e.g., Enbrel, Remicade, Humira, and efalizumab), monoclonal antibodies against B cells (e.g., Rituxan); T cell activation blockers (e.g., Orencia), IL-1 blockers (e.g., Anakinra), coal tar, herbal remedies (e.g., U.S. Pat. No. 6,403,654 and U.S. Pat. No. 6,153,197), and phototherapy. Additionally, some treatments with different modes of action may be used in combination with improved results. For example, application of an ointment comprising calcipotriol, a vitamin D₃ analog, and betamethasone dipropionate, a corticosteroid, was more efficacious than an ointment comprising calcipotriol alone (Kragballe et al. 2004). In particular, it may be necessary to consider a combination of therapies if a subject has more than one type of psoriasis or if the disease affects more than 5% of the BSA (Pariser et al. 2007).

However, these treatments suffer from various disadvantages including cosmetic liabilities, severe side effects, high cost, and minimal or short-term efficacy. Highlighting the need for new therapies is a survey which found that nearly half of the psoriasis patients who responded were dissatisfied with current treatment options (Kreuger et al. 2001).

Meisoindigo, a derivative of Indigo naturalis, has been examined for the treatment of psoriasis via oral ingestion (Lin X Q 1989; Caixia 1991). The optimal dose of meisoindigo in the treatment of psoriasis was reported to be 150 mg (50 mg, 3 times a day). This dosage level of meisoindigo has been studied in the treatment of malignant disease, i.e. chronic myelogenous leukemia, in China at which various side effects have been reported, including bone, joint, and muscle pain of various degrees (45.08% of patients suffered from sharp, stabbing, or burning pain), and gastrointestinal problems (45.87% of patients suffered from nausea, vomiting, abdominal pain, distension, and/or diarrhea). Other, less frequent, side effects included bone marrow suppression (0.4%), mild facial edema (6.7%), and dizziness with temporary memory loss (0.2%) (Xiao et al. 2002). These side effects make meisoindigo an undesirable oral medication for the treatment of psoriasis. Moreover, due to poor solubility in both oil and aqueous phases and other unfavorable physical and chemical properties, meisoindigo has only been available in tablet form.

The inventors developed a novel composition designed for topical application comprising meisoindigo as the active ingredient, and surprisingly discovered that the composition effectually treated psoriasis without producing any of the negative side effects associated with oral administration as described in the prior art. In fact, the inventors discovered that treatment of psoriasis with topical meisoindigo achieved a degree of efficacy similar to a potent corticosteroid. Thus, the presently disclosed method provides a means of treating psoriasis that is cosmetically acceptable, effective, and easy to apply.

### SUMMARY OF THE INVENTION

The present invention is directed to a topical psoriasis medicament or composition. The topical composition preferably comprises a therapeutically effective amount of meisoindigo and a pharmaceutically acceptable topical carrier. In certain embodiments, the topical composition may also further comprise one or more additional active agents to treat psoriasis or provide a synergistic effect with meisoindigo.

The present invention is also directed to methods of treating psoriasis. The method preferably comprises the steps of topically applying the topical psoriasis medicament or composition to an affected epidermal area of a subject.

Preferably the subject is human and the topical composition is applied at least twice a day to the affected epidermal area of the subject.

### BRIEF DESCRIPTION OF THE DRAWINGS

FIG. 1 illustrates the chemical structure of the primary active ingredient, meisoindigo; and

FIG. 2 is a line graph illustrating the linear relationship between meisoindigo concentration and peak area as determined by high performance liquid chromatography.

FIG. 3 is a photograph of an affected epidermal area of Patient No. 2 in Example 3.2, taken at the eligibility visit.

FIG. 4 is a photograph of the same affected epidermal area of Patient No. 2 in Example 3.2, taken at the four-week visit (Visit 2).

FIG. 5 is a photograph of an affected epidermal area of Patient No. 5 in Example 3.2, taken at the eligibility visit.

FIG. 6 is a photograph of the same affected epidermal area of Patient No. 5 in Example 3.2, taken at the four-week visit (Visit 2).

### DETAILED DESCRIPTION OF THE PREFERRED EMBODIMENTS

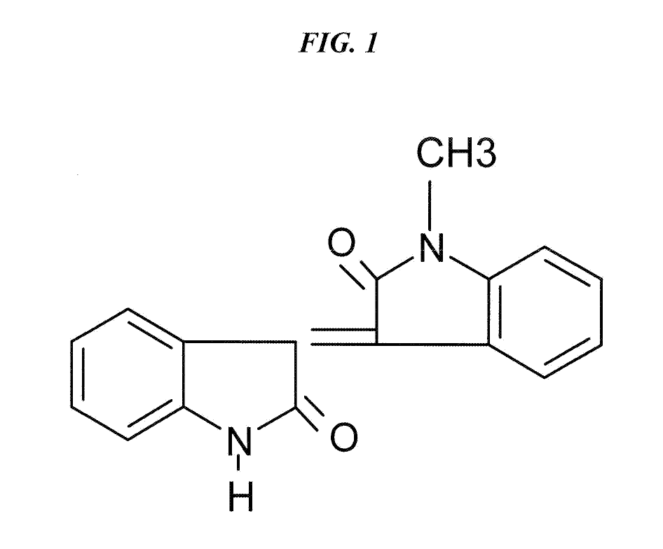

The present invention is directed to topical compositions and methods of use thereof for treating psoriasis. The topical compositions preferably comprise meisoindigo as the primary active ingredient. As used herein, “meisoindigo” refers to methylated isoindigo and analogs, derivatives, or metabolites thereof, e.g., N-methyl-Δ3,3′-dihydroindole-2,2′ diketone as illustrated in FIG. 1. Specifically, the disclosed topical composition/medicament may be a cream, lotion, spray, oil, ointment, paste, dressing, solution, or other type of composition that lends itself to topical application.

In a first embodiment, the invention is directed to a topical composition for treating psoriasis comprising a therapeutically effective amount of meisoindigo and a pharmaceutically acceptable carrier. A “therapeutically effective amount” is an amount necessary to palliate at least one symptom of psoriasis. For example, a therapeutically effective amount is sufficient to treat (i.e. alleviate or reduce) at least one of: itching/scratching, redness, inflammation, cracking, scaling, bleeding, etc. Preferably, the therapeutically effective amount of meisoindigo comprises between 0.001 to 5.00% by weight of the composition, more preferably 0.10 to 1.50%, and most preferably 0.10 to 0.70%. In another preferred embodiment, meisoindigo refers to the chemical compound N-methyl-Δ3,3′-dihydroindole-2,2′ diketone illustrated in FIG. 1.

The term “pharmaceutically acceptable” means approved by a regulatory agency of the Federal or state government or listed in the U.S. Pharmacopeia or other generally recognized pharmacopeia for use in animals, and more particularly in humans. The term “carrier” refers to a diluent, adjuvant, excipient, penetration enhancer, or vehicle with which an active ingredient is administered. Such pharmaceutical carriers can be liquids, such as water and oils, including those of petroleum, animal, vegetable or synthetic origin. Preferably, the pharmaceutically acceptable carrier comprises excipients commonly used in topically applied formulations (water, oil-based lotions, sprays, ointments, etc.). As a non-limiting example, a pharmaceutically acceptable carrier may comprise water, glycerin, petrolatum, stearic acid, glycol stearate, dimethicone, isopropyl isostearate, tapioca starch, cetyl alcohol, glyceryl stearate, magnesium aluminum silicate, carbomer, ethylene brassylate, triethanolamine, disodium EDTA, phenoxyethanol, methyl paraben, propyl paraben, ethanol, bio-polymers (e.g., sodium hyaloronate), liposomes, nano- and micro-particulate carriers, and/or titanium dioxide. More preferably, the pharmaceutically acceptable carrier comprises dimethyl sulfoxide (DMSO), glycerol, propylene glycol, petrolatum water, and one or more pharmaceutically acceptable penetration enhancer (absorption promoter and/or accelerants).

In a preferred embodiment, the composition comprises DMSO between 0.10 to 5.00% by weight, and more preferably 0.20 to 1.00%. However, some patients may harbor an allergy to DMSO, in which case the composition preferably comprises glycerol or propylene glycol between 1.00 and 10.00% by weight, more preferably 2.00 to 6.00%, and most preferably 2.00 to 3.00%.

In another embodiment, the topical compositions are in the form of oil-in-water (O/W) or water-in-oil (W/O) creams, lotions, or ointments. The oily phase may be a vegetable oil (e.g, olive oil, arachis oil, etc.), a mineral oil (e.g., liquid paraffin), or mixtures of both. Suitable emulsifying agents may be naturally-occurring gums (e.g., acacia, gum tragacanth, etc.), naturally-occurring phosphatides (e.g., soy bean, lecithin, esters or partial esters derived from fatty acids and hexitol, etc), anhydrides (e.g., sorbitan monooleate), and condensation products of said partial esters with ethylene oxide (e.g., polyoxyethylene sorbitan monooleate). These emulsions may also contain coloring and flavoring agents. Typically, the topical compositions of the invention comprise meisoindigo, skin penetration enhancers, pharmaceutical surfactants and solubility enhancers, oil phase components, aqueous phase components, emulsifiers, moisturizers, antioxidants, vitamins, lubricants, preservatives, and other ingredients.

Skin penetration enhancers reversibly decrease the barrier resistance of the skin, which increases the amount of meisoindigo absorbed. Preferably, skin penetration enhancers include, but are not limited to, sulfoxides (e.g. DMSO), azones (e.g. laurocapram), pyrrolidones (e.g., 2-pyrrolidone), alcohols and alkanols (e.g., ethanol, decanol, etc.), oleic acid (and derivatives thereof), glycols (e.g., propylene glycol), dimethylformamide (DMF), dimethylacetamide (DMAC), fatty alcohols (e.g., lauryl alcohol), fatty acid esters, fatty acids, fatty alcohol ethers (e.g., EO-2-oleyl ether), terpenes, and biologics (e.g., lecithin). Skin penetration enhancers preferably comprise 0.01 to 10.00% by weight of the composition, and more preferably 0.01 to 5.00%.

Pharmaceutical surfactants or solubility enhancers include, but are not limited to, lauryl alcohol, polyoxyethylene ether, polyoxyethylene glycerol monostearate, stearic acid ester oxygen poly hydrocarbon, vitamin E succinate polyethylene glycol ester, polyoxyethylene castor oil, polyoxyethylene hydrogenated castor oil, poloxamer, organic esters (e.g. ethylene acetate), and poly hill dinitrate 80 (i.e. Tween 80 or its mixture). In a preferred embodiment, the pharmaceutical surfactants or solubility enhancers include lauryl alcohol polyoxyethylene 23 ether, stearic acid hydrocarbon oxygen Poly (40) ester, polyoxyethylene (35) castor oil, poloxamer-F127, and poloxamer F-68. Still, in a most preferred embodiment, the pharmaceutical surfactants or solubility enhancers include DMSO, polyvinylpyrrolidone (K17, K25, K30, K90), PEG 400, 4000, and 6000 (and their mixtures), stearic acid hydrocarbon oxygen Poly (40) ester, lauryl alcohol polyoxyethylene (23) ether, vitamin E succinate polyethylene glycol ester, ethylene acetate, and polyoxyethylene (40) hydrogenated castor oil (and its mixtures, i.e. polyoxy (40) stearate). These pharmaceutical surfactants or solubility enhancers preferably comprise 0.01 to 10.00% by weight of the composition, and more preferably 0.01 to 5.00%.

Oil phase components include, but are not limited to, glyceryl monoacetate, glycerol diacetate, glyceryl triacetate, stearic acid, soybean oil, corn oil, peanut oil, palmitic acid, palm oil, sunflower oil, olive oil, coconut oil, sesame oil, cotton seed oil, low erucic acid rapeseed oil, oleic acid, medium-chain triglycerides, single-decane triglyceride, animal fat (e.g., lanolin), beeswax, petrolatum, hydrocarbons, and Vaseline. Oil phase components preferably comprise 1.00 to 85.00% by weight of the composition, and more preferably 1.00 to 80.00%.

Aqueous phase components include, but are not limited to, de-ionized water, glycerol gelatin, cellulose derivatives (e.g., microcrystalline cellulose (Avicel PH 101)), and polyethylene glycol (PEG 300 to PEG 6000). Aqueous phase components preferably comprise 1.00 to 85.00% by weight of the composition, and more preferably 1.00 to 80.00%.

Emulsifiers include, but are not limited to, polyoxyethylene oleyl ether, PEG-40 stearate, ceteareth-12, Eumulgin B-1 (Henkel), ceteareth-20, Eumulgin B-2 (Henkel), ceteareth-30, Lanette O (Henkel), glyceryl stearate Cutina GMS (Henkel), PEG-100 stearate, methyl myristate, isopropyl myristate, Arlacel 165, glyceryl stearate, PEG-100 stearate, steareth-2 and steareth-20, dimethicone copolyol, Polysorbate 20 (Tween 20), Polysorbate 40 (Tween 40), Polysorbate 60 (Tween 60), Polysorbate 80 (Tween 80), lauramide DEA, cocamide DEA, and cocamide MEA, Phospholipid PTC, alginate, carrageenan, Glucate DO, methylcellulose, polyvinyl alcohol, Cocamidopropyl phosphatidyl PG-dimonium chloride, Pemulen TR 1, Pemulen TR 2, Carbopol 1342, Carbopol 1382, Carbomer 1342, Carbomer 934, Carbomer 934P, Carbomer 940, Carbomer 941, Carbomer 974P, Carbomer 980, and Carbomer 981. Preferably, emulsifiers are selected from the group consisting of stearic acid, magnesium stearate, milk amino acids, sodium lauryl sulfate, triethanolamine, and magnesium aluminum silicate. Emulsifiers preferably comprise 0.01 to 10.00% by weight of the composition, and more preferably 0.01 to 5.00%.

Moisturizers include, but are not limited to, glycerol, propylene glycol, and sorbitol. Moisturizers preferably comprise 0.50 to 25.00% by weight of the composition, more preferably 1.00 to 20.00%, and most preferably 2.00 to 10.00%.

Antioxidants include, but are not limited to, water soluble antioxidants, lipid-soluble antioxidants, vitamin C, vitamin C palmitate, propyl gallate, vitamin E (tocopherol), tert-butyl ether-hydroxybenzoate fennel, 2,6 di-tert-butyl-p-cresol, or mixtures of one or more antioxidants. Preferably, antioxidants include vitamin C, vitamin C palmitate, propyl gallate, vitamin E (tocopherol), tert-butyl ether-hydroxybenzoate fennel. Most preferably antioxidants include vitamin C palmitate.

Antioxidants preferably comprise 0.01 to 10.00% by weight of the composition, and more preferably 0.01 to 5.00%.

Vitamins include, but are not limited to, vitamin A, vitamin B series, vitamin C, vitamin D, vitamin E. Vitamins preferably comprise 0.01 to 10.00% by weight of the composition, and more preferably 0.01 to 5.00%.

Lubricants include, but are not limited to, urea, magnesium stearate, sodium lauryl sulfate, polyethylene glycol, and silica gel powder. Lubricants preferably comprise 0.01 to 10.00% by weight of the composition, and more preferably 0.01 to 5.00%.

Preservatives include, but are not limited to, chloro-m-cresol, citric acid, disodium edetate, ethoxylated alcohol, glycerin, 1,2,6-hexanetriol, methylparaben, parabens, potassium, sorbate, propyl gallate, propylene glycol, propyl paraben, sodium bisulfate, sodium citrate, butyl paraben, sodium metabisulfite, sorbic acid, tannic acid, zinc stearate, butylated hydroxytoluene, butylated hydroxyanisole, benzoic acid, salicylic acid, propyl paraben, dichlorobenzyl alcohol, formaldehyde, alpha-tocopherol, sodium ascorbate, ascorbic acid, ascorbyl palmitate phenol, m-cresol, bisphenol, cetrimide, benzalkonium chloride, sorbic acid, polyquatemum-1, chlorobutanol, chlorhexidine, Dowcell 200 (Dow Chemical Co., Midland, Mich.), Glydant (dimethylol-25,5-dimethylhydantoin, Lonza, Inc, Fairlawn, N.J.), Germal 115 (imidazolidylurea, Sutton Laboratories, Chatham, N.J.), Germal II (diazolidinylurea, Sutton), sodium hydroxymethylglycinate, Buzan 1504 (dimethhydroxymethylpyrazole, Buckman Labs, Memphis, Tenn.), phenoxyethanol, and benzoyl peroxide. Preferably, preservatives are selected from the group consisting of hydroxylethyl benzene, hydroxylmethyl benzene, phenoxyethanol, propyl paraben, and methyl paraben. Preservatives preferably comprise 0.01 to 10.00% by weight of the composition, and more preferably 0.05 to 5.00%.

Other ingredients may include acid buffers (e.g., buffered solutions of acetic acid, formic acid, phosphoric acid, boric acid, citric acid, and ascorbic acid) and hydrophilicity modulators (e.g., polymers of 2-acrylamido-2-methylpropanesulfonic acid, alkyl sulfates, aryl sulfates, alkyl sulfonates, and aryl sulfonates). Preferably however, other ingredients are selected from the group consisting of keratin, collagen, amino acids, lecithin, aloe extracts, dimethicone, and disodium EDTA. These other ingredients preferably comprise 0.01 to 20.00% by weight of the composition, more preferably 0.01 to 10.00%, and most preferably 0.01 to 5.00%.

In another embodiment, the topical composition further comprises an additional agent or agents commonly used in the treatment of psoriasis via topical application. Increased efficacy may be achieved by combining agents or therapies with different modes of action for treating the symptoms of psoriasis, and thus, such combinations are preferred. In vitro experiments suggest that meisoindigo rebalances the cytokine profile by inhibiting expression of pro-inflammatory cytokines such as IL-10, IL-6, IL-17, and TNF-\acute{α}, thereby selectively inhibiting Stat3 signaling while stimulating expression of the anti-inflammatory cytokine IL-10. Additionally, meisoindigo inhibits differentiation naïve T cells into Th1 and Th17, restoring normal T cell function in inflamed skin tissue (Glatigny et al. 2010). Therefore, in a further preferred embodiment, the additional agents are selected from the group consisting of coal tar, calcipotriol (calcipotriene), corticosteroids, retinoids, herbal remedies (e.g., aloe extracts), analgesics, and nonsteroidal anti-inflammatory drugs (e.g., aspirin, salicylic acid, etc.).

In yet another embodiment, the invention is directed to a method of treating psoriasis comprising the steps of topically applying a composition comprising meisoindigo within a pharmaceutically acceptable carrier to an affected epidermal area of a subject suffering from psoriasis. As used herein, “affected epidermal area” refers to those patches of skin that exhibit common indicators of psoriasis including inflammation, hyperproliferation, cracking, scaling, and bleeding.

Furthermore, the method is directed to treating a type of psoriasis selected from the group consisting of plaque psoriasis, guttate psoriasis, inverse psoriasis, pustular psoriasis, and erythrodermic psoriasis. Most preferably, the method is directed to treating plaque psoriasis. Additionally, the subject suffering from psoriasis is an animal, preferably a mammal (e.g., pig, cow, horse, dog, cat, etc.), and most preferably the subject is a human.

In studies conducted thus far (see Example 3), no side effects have been observed. Thus, the methods disclosed herein represent a very desirable first- or second-line therapy for the treatment of psoriasis in those patients exhibiting a mild form of the disease. However, the methods have also been demonstrated to be quite effective at treating the moderate to severe forms of psoriasis. Thus, in a preferred embodiment, the method is directed to the treatment of mild, moderate, or severe psoriasis, and more preferably directed to the treatment of moderate or severe psoriasis.

In another embodiment, the method comprises the steps of topically applying a composition comprising meisoindigo and an additional agent or agents commonly used in the treatment of psoriasis via topical application. Preferably, the additional agents commonly used in the treatment of psoriasis via topical application are selected from the group consisting of coal tar, calcipotriol (calcipotriene), corticosteroids, retinoids, herbal remedies (e.g., aloe extracts), analgesics, and nonsteroidal anti-inflammatory drugs (e.g., aspirin, salicylic acid, etc.).

Moreover, the disclosed method may be combined with other common therapies used in the treatment of psoriasis. These other therapies typically comprise oral ingestion, infusion, or injection of a compound that has been demonstrated to palliate symptoms of psoriasis. Such compounds include DMARDs, TNF-\acute{α} blockers, monoclonal antibodies against B cells, T cell activation blockers, IL-1 blockers, and herbal remedies. Furthermore, phototherapy is also a common method employed to treat psoriasis.

Thus, in another embodiment, the invention discloses a method of treating psoriasis comprising the steps of topically, applying a composition comprising meisoindigo with or without an additional agent or agents commonly used in the treatment of psoriasis via topical application, in conjunction with another common psoriatic therapy. Preferably, another common psoriatic therapy is selected from the group consisting of administration of DMARDs, TNF-\acute{α} blockers, monoclonal antibodies against B cells, T cell activation blockers, IL-1 blockers, herbal remedies, and phototherapy. Most preferably, another common therapy is selected from administration of the group consisting of alefacept, efalizumab, infliximab, adalimumab, etanercept, ustekinumab, methotrexate, cyclosporine, and phototherapy.

The method can be employed as needed, preferably after bathing or gently washing the affected area. In a preferred embodiment, the method comprises topically applying the composition one to four times per day, and most preferably twice a day (morning and night). In another embodiment the treatment period (i.e. the length of time for which a subject has applied the composition on a daily basis) lasts until the subject is satisfied with the comfort and appearance of the treatment area. Preferably, the treatment period does not exceed six months. More preferably, the treatment period does not exceed three months.

### EXAMPLES

The present invention will now be illustrated by the following examples. It is to be understood that the ensuing examples are for exemplary purposes only and are not intended to limit the scope of the invention. One skilled in the art can appreciate that modification may be made without departing from the spirit or scope of the present invention as set forth in the claims. Particularly, advances in the state of the art (e.g., Avestin C50 microfluidizer) may be used in compositions and medicaments comprising meisoindigo.

### Example 1

### Formulations of Meisoindigo Creams/Lotions, Gels and Sprays for the Treatment of Psoriasis

Structurally, meisoindigo is a heterocyclic compound, and therefore, is poorly soluble in both oil and aqueous phases. Thus, the inventors designed different formulation procedures, and varieties of excipients of oil and aqueous phases, surfactants and solubility enhancers, and emulsifiers in order to develop stable, uniform, and cosmetically acceptable creams. Meisoindigo, Lot#NAT-0601, was synthesized under US cGMP conditions and chemical identity was confirmed by melting point, ultraviolet spectroscopy, nuclear magnetic resonance spectroscopy (NMR), and mass spectrometry. Purity was established at 100.0% with high performance liquid chromatography (HPLC). The meisoindigo was produced on Jun. 2, 2006, retested in July 2008, and expired in July 2009.

Emulsifiers and/or surfactants were used not only to emulsify, but also to enhance the solubility of meisoindigo. Because a surfactant contains both hydrophilic and lipophilic groups, it can surround both the water- and oil-soluble regions of the meisoindigo molecule, resulting in enhanced solubility and increased dissolution and distribution of the meisoindigo molecules in the cream or lotion.

Lipids and vitamins, such as Vitamin E, were used as diluents or cosolvents of meisoindigo. Skin penetration enhancers were added to increase absorption of meisoindigo in order to achieve sufficient bioavailability. Moisturizers were used to protect the skin and to ameliorate the dryness and flakiness associated with psoriasis. Antioxidants and preservatives were used to prevent oxidation and microbial damage to the other compounds in the cream or lotion.

The formulations below are topical formulations containing meisoindigo as an active ingredient to be used to treat psoriasis without notable side effects.

### Example 1.1

### Formulation 1

| Cream Base | | |
|---|---|---|
| Stearic acid | 12.00 g | (1) |
| Monostearin (monoglyceride) | 3.50 g | (2) |
| Liquid paraffin | 6.00 g | (3) |
| Lanolin | 5.00 g | (4) |
| Vaseline | 1.00 g | (5) |
| Propyl paraben | 0.25 g | (6) |
| Methyl paraben | 0.10 g | (7) |
| Triethanolamine | 0.40 g | (8) |
| De-ionized water | 70.25 g | (9) |
| Primary Active Ingredient | | |
| Meisoindigo | 0.50 g | (10) |
| Other Ingredients | | |
| Dimethyl Sulfoxide (DMSO) | 1.00 g | (11) |

### Procedures

Components (1) through (5) were melted by heating to 80° C., and then filtered and maintained at 80° C. in a water bath to which components (6) and (7) were added, and dissolved to form an oil phase solution.
Component (8) was dissolved in de-ionized water (9), and filter-sterilized (aqueous phase). The aqueous phase solution was then heated and maintained at 80° C. in a water bath until use.
Oil phase was slowly added to aqueous phase with strong stirring until it was cooled down to a room temperature, forming a cream base (Cream Base 1).
Suitable amount of Meisoindigo (10) was dissolved in DMSO (11) in a mortar to which cream base was added and triturated deliberately to obtain Cream Formulation 1.
   Comments: The presence of ions, such as Ca⁺⁺, Mg⁺⁺, as well as acidic or alkalized conditions, significantly decreases stability of this formulation, and should be avoided.

### Example 1.2

### Formulation 2

| Cream base | | |
|---|---|---|
| Stearic acid | 12.00 g | (1) |
| Monostearin (monoglyceride) | 3.50 g | (2) |
| Liquid paraffin | 6.00 g | (3) |
| Lanolin | 5.0 g | (4) |
| Vaseline | 1.00 g | (5) |
| Propyl paraben | 0.25 g | (6) |
| Methyl paraben | 0.10 g | (7) |
| Triethanolamine | 0.40 g | (8) |
| disodium EDTA | 0.04 g | (9) |
| De-ionized water | 67.71 g | (10) |
| Primary Active Ingredient | | |
| Meisoindigo | 0.50 g | (11) |
| Other Ingredients | | |
| Dimethyl Sulfoxide (DMSO) | 0.50 g | (12) |
| Glycerol | 3.00 g | (13) |

### Procedures

Components (1) through (5) were melted and sterilized by heating to 80° C., and then filtered and maintained at 80° C. in a water bath to which components (6) and (7) were added, and dissolved to form an oil phase solution.
Components (8) and (9) were dissolved in de-ionized water (10), pH was adjusted to 7.0 with either 0.01N HCl or 0.01N NaOH and filter-sterilized (aqueous phase solution). The aqueous solution was heated and kept in a water bath at 70° C. until use.
Oil phase was then added to aqueous phase with strong stirring to form a vanishing base (Cream Base 2) and the cream was cooled down to a room temperature.
Suitable amount of Meisoindigo (11) was dissolved in DMSO (12) and glycerol (13) in a mortar to which cream base was added and triturated deliberately to obtain Cream Formulation 2.
   Comments: To avoid destabilization by ions and protect the skin from dryness, disodium EDTA and glycerol were added to the aqueous phase for enhanced skin protection.

### Example 1.3

### Formulation 3

| Cream Base | | |
|---|---|---|
| Stearic acid | 12.00 g | (1) |
| Monostearin (monoglyceride) | 3.50 g | (2) |
| Liquid paraffin | 6.00 g | (3) |
| Lanolin | 5.0 g | (4) |
| Vaseline | 1.00 g | (5) |
| Vitamin A palmitate | 1.00 g | (6) |
| Propyl paraben | 0.25 g | (7) |
| Methyl paraben | 0.10 g | (8) |
| Triethanolamine | 0.40 g | (9) |
| Disodium EDTA | 0.04 g | (10) |
| De-ionized water | 60.96 g | (11) |
| Primary Active Ingredient | | |
| Meisoindigo | 0.50 g | (12) |
| Other Ingredients | | |
| Dimethyl Sulfoxide (DMSO) | 0.25 g | (13) |
| Aloe extracts in water | 5.00 g | (14) |
| Vitamin E | 1.00 g | (15) |
| Glycerol | 3.00 g | (16) |

### Procedures

Components (1) through (5) were melted and sterilized by heating to 80° C., filtered and maintained at 80° C. in a water bath to which components (6), (7), and (8) were added, and mixed evenly to form an oil phase solution.
Components (9) and (10) were dissolved in de-ionized water (11), pH was adjusted to 7.0 with either 0.01N HCl or 0.01N NaOH and filter-sterilized (aqueous phase solution). The aqueous solution was heated and kept in a water bath at 80° C. until use.
Oil phase was then added to aqueous phase with strong stirring to form a cream base (Cream Base 3) and cooled down to room temperature.
Suitable amount of Meisoindigo (12) was dissolved in DMSO (13) and Vitamin E (15) in a mortar to which glycerol (16) and aloe extract solution (14) were sequentially added, followed by the cream base. The mixture was triturated deliberately to obtain Cream Formulation 3.
   Comments: To protect the skin from dryness, Cream Formulation 3 was furthered improved by adding aloe extracts, Vitamins A and E, as well as glycerol, which made the cream more amenable to use in the treatment of psoriasis. Since DMSO may cause an allergic reaction, the amount of DMSO was reduced in this formulation.

### Example 1.4

### Formulation 4

| Cream Base | | |
|---|---|---|
| White beeswax | 18.00 g | (1) |
| Almond oil | 31.00 g | (2) |
| Liquid paraffin | 30.00 g | (3) |
| Propyl paraben | 0.25 g | (4) |
| Methyl paraben | 0.10 g | (5) |
| Borax | 1.0 g | (6) |
| De-ionized water | 17.15 g | (7) |
| Primary Active Ingredient | | |
| Meisoindigo | 0.50 g | (8) |
| Other Ingredients | | |
| Glycerol | 2.00 g | (9) |

### Procedures

Components (1) through (5) were melted by heating to 70° C., filter-sterilized, and then maintained at 70° C. in a water bath until use (oil phase solution).
Component (6) was dissolved in de-ionized water (7) by heating at 70° C. and filter-sterilized to obtain aqueous phase. The aqueous phase was maintained in a water bath at 70° C. until use.
Oil phase was then added to aqueous phase with strong stirring, forming a W/O cream base (Cream Base 4) and cooled down to room temperature.
Suitable amount of Meisoindigo (8) was added to glycerol (9) in a mortar and triturated deliberately. W/O cream base was then added and continuously triturated to obtain Cream Formulation 4.
   Comments: This cream contains a high percentage of oil, thus it is a water-in-oil (W/O) cream. The high oil content effectively blocks the loss of moisture from the skin. Since DMSO may cause allergic reaction, the glycerol was used to replace DMSO to enhance the solubility of meisoindigo and protect the skin from dryness.

### Example 1.5

### Formulation 5

| Cream Base | | |
|---|---|---|
| White beeswax | 18.00 g | (1) |
| Almond oil | 30.00 g | (2) |
| Liquid paraffin | 29.00 g | (3) |
| Vitamin A | 1.00 g | (4) |
| Propyl paraben | 0.25 g | (5) |
| Methyl paraben | 0.10 g | (6) |
| Aloe extracts in water | 2.00 g | (7) |
| Borax | 1.0 g | (8) |
| De-ionized water | 15.15 g | (9) |
| Primary Active Ingredient | | |
| Meisoindigo | 0.50 g | (10) |
| Other Ingredients | | |
| Vitamin E | 1.00 g | (11) |
| Glycerol | 2.00 g | (12) |

### Procedures

Components (1) through (6) were melted by heating to 70° C., filter-sterilized, and maintained at 70° C. in a water bath until use (oil phase solution).
Component (8) was dissolved in de-ionized water (9) by heating to 70° C. and mixed evenly with component (7). This mixture was filter-sterilized (aqueous phase solution) and maintained in a water bath at 70° C. until use.
Oil phase solution was then added to aqueous phase solution with strong stirring to form a W/O cream base (Cream Base 5) and cooled down to room temperature.
Suitable amount of Meisoindigo (10) was added to Vitamin E (11) and glycerol (12) in a mortar and triturated deliberately. The W/O cream base was then added and continuously triturated to obtain Cream Formulation 5.
   Comments: Cream Formulation 5 was enhanced by adding Vitamins A and E, as well as aloe extract, which make the cream more amenable for use in the treatment of psoriasis when itchiness and dryness are bothersome.

### Example 1.6

### Formulation 6

| Cream Base | | |
|---|---|---|
| White beeswax | 9.00 g | (1) |
| Almond oil | 16.00 g | (2) |
| Liquid paraffin | 15.00 g | (3) |
| Propyl paraben | 0.25 g | (4) |
| Methyl paraben | 0.10 g | (5) |
| Borax | 1.0 g | (6) |
| De-ionized water | 56.15 g | (7) |
| Primary Active Ingredient | | |
| Meisoindigo | 0.50 g | (8) |
| Other Ingredients | | |
| Glycerol | 2.00 g | (9) |

### Procedures

Components (1) through (5) were melted by heating to 70° C., filter-sterilized, and then maintained at 70° C. in a water bath until use (oil phase solution).
Component (6) was dissolved in de-ionized water (7) and filter-sterilized to obtain aqueous phase solution. The aqueous phase solution was heated in a water bath at 70° C. until use.
Oil phase solution was then added to aqueous phase solution with strong stirring and an oil-in-water (O/W) cream base (Cream Base 6) was formed and cooled down to room temperature.
Suitable amount of Meisoindigo (8) was added to glycerol (9) in a mortar and triturated deliberately. The O/W cream base was then added and continuously triturated to obtain Cream Formulation 6.
   Comments: The water-in-oil (W/0) cream of Formulation 4 can be converted to an oil-in-water (O/W) cream by changing the ratio of oil/water as shown in Formulation 6 above. Formulation 6 is advantageous in the treatment of psoriasis when itchiness and dryness are bothersome.

### Example 1.7

### Formulation 7

| Cream Base | | |
|---|---|---|
| White beeswax | 9.00 g | (1) |
| Almond oil | 16.00 g | (2) |
| Liquid paraffin | 15.00 g | (3) |
| Vitamin A | 1.00 g | (4) |
| Propyl paraben | 0.25 g | (5) |
| Methyl paraben | 0.10 g | (6) |
| Aloe extracts in water | 2.00 g | (7) |
| Borax | 1.0 g | (8) |
| De-ionized water | 53.15 g | (9) |
| Primary Active Ingredient | | |
| Meisoindigo | 0.50 g | (10) |
| Other Ingredients | | |
| Vitamin E | 1.00 g | (11) |
| Glycerol | 2.00 g | (12) |

### Procedures

Components (1) through (6) were melted by heating to 70° C., filter-sterilized, and then maintained at 70° C. in a water bath until use (oil phase).
Component (8) was dissolved in de-ionized water (9) and mixed evenly with component (7). This mixture was filter-sterilized (aqueous phase solution) and maintained in a water bath at 70° C. until use.
Oil phase solution was then added to aqueous phase solution with strong stirring, and an O/W cream base (Cream Base 7) was obtained, and cooled to room temperature.
Suitable amount of Meisoindigo (10) was added to Vitamin E (11) and glycerol (12) in a mortar and triturated deliberately. The O/W cream base was then added and continuously triturated to obtain Cream Formulation 7.
   Comments: Cream Formulation 7 was enhanced by adding Vitamins A and E, as well as aloe extract and glycerol to moisturize the skin, which makes the cream more amenable to use in the treatment of psoriasis when itchiness and dryness are bothersome.

### Example 1.8

### Formulation 8

| Cream Base | | |
|---|---|---|
| Octadecanol | 25.00 g | (1) |
| White Vaselin | 25.00 g | (2) |
| Propyl paraben | 0.25 g | (3) |
| Methyl paraben | 0.10 g | (4) |
| Dodecanol sulfate | 1.00 g | (5) |
| Propylene glycol | 10.0 g | (6) |
| De-ionized water | 53.15 g | (7) |
| Primary Active Ingredient | | |
| Meisoindigo | 0.50 g | (8) |
| Other Ingredients | | |
| Propylene glycol | 2.00 g | (9) |

### Procedures

Components (3) and (4) were dissolved in component (6) (solution 1). Components (1) and (2) were mixed and melted by heating to 70° C., thereafter solution 1 was added and mixed evenly. This mixture was filter-sterilized, and then maintained at 70° C. in a water bath until use (oil phase solution).
Component (5) was dissolved in de-ionized water (7), and filter-sterilized (aqueous phase solution). The aqueous phase solution was maintained in a water bath at 70° C. until use.
Oil phase was then added to aqueous phase with strong stirring, and an O/W cream base (Cream Base 8) was obtained and cooled to room temperature.
Suitable amount of Meisoindigo (8) was added to propylene glycol (9) in a mortar and triturated deliberately. The O/W cream base was then added and continuously triturated to obtain Cream Formulation 8.
   Comments: Propylene glycol in this formulation serves as a moisturizer, solubilizer of meisoindigo, preservative, and skin penetration enhancer. This formulation serves to moisturize the skin, which makes Cream Formulation 8 more amenable to use in the treatment of psoriasis when itchiness and dryness are problematic.

### Example 1.9

### Formulation 9

| Cream Base | | |
|---|---|---|
| Octadecanol | 25.00 g | (1) |
| White Vaselin | 25.00 g | (2) |
| Propyl paraben | 0.25 g | (3) |
| Methyl paraben | 0.10 g | (4) |
| Dodecanol sulfate | 1.00 g | (5) |
| Vitamin A | 1.00 g | (6) |
| Aloe extracts in water | 2.00 g | (7) |
| Propylene glycol | 10.0 g | (8) |
| De-ionized water | 50.15 g | (9) |
| Primary Active Ingredient | | |
| Meisoindigo | 0.50 g | (10) |
| Other Ingredients | | |
| Propylene glycol | 2.00 g | (11) |
| Vitamin E | 1.00 g | (12) |

### Procedures

Components (3), (4), (6) were dissolved in propylene glycol (8) (solution 1). Components (1) and (2) were mixed and melted by heating to 70° C., thereafter solution 1 was added and mixed evenly. This mixture was filter-sterilized and maintained at 70° C. in a water bath until use (oil phase solution).
Component (5) was dissolved in de-ionized water (9), to which aloe extract (7) was added, and mixed evenly. The solution was filter-sterilized to obtain the final aqueous phase solution. The aqueous phase solution was maintained in a water bath at 70° C. until use.
Oil phase solution was then added to aqueous phase solution with strong stirring, and an O/W cream base (Cream Base 9) was obtained and cooled to room temperature.
Suitable amount of Meisoindigo (10) was added to propylene glycol (9) and vitamin E (12) in a mortar and triturated deliberately. The O/W cream base was then added and continuously triturated to obtain Cream Formulation 9.
   Comments: Formulation 9 was therapeutically enhanced by adding Vitamins A and E, as well as aloe extract to moisturize the skin, which makes Cream Formulation 9 amenable to use in the treatment of psoriasis when itchiness and dryness are bothersome.

### Example 1.10

### Formulation 10

| Cream Base | | |
|---|---|---|
| Stearic acid | 15.00 g | (1) |
| White Vaselin | 10.00 g | (2) |
| Monostearin | 8.50 g | (3) |
| Propyl paraben | 0.28 g | (4) |
| Methyl paraben | 0.11 g | (5) |
| Glycerol | 5.50 g | (6) |
| Tween 80 | 3.00 g | (7) |
| De-ionized water | 55.11 g | (8) |
| Primary Active Ingredient | | |
| Meisoindigo | 0.50 g | (9) |
| Other Ingredients | | |
| Glycerol | 2.00 g | (10) |

### Procedures

Components (1) through (5) were melted by heating at 70° C., and mixed evenly. This mixture was filter-sterilized, and then maintained at 70° C. in a water bath until use (oil phase solution).
Components (6) and (7) were mixed evenly with de-ionized water (8). This solution was filter-sterilized to obtain the final aqueous phase solution. The aqueous phase solution was maintained in a water bath at 70° C. until use.
Oil phase solution was then slowly added to aqueous phase solution with strong stirring, and an O/W cream base (Cream Base 10) was obtained and cooled to room temperature.
Suitable amount of Meisoindigo (9) was added to glycerol (10) in a mortar and triturated deliberately. The O/W cream base was then added and continuously triturated to obtain Cream Formulation 10.
   Comments: Tween 80 in this formulation serves as an emulsifer and solubilizer, and allows for the inclusion of a greater amount of meisoindigo, which is acidic. For example, up to 5 g (5%, w/w) of meisoindigo may be included in this, or a similar, formulation. However, Tween 80 may reduce the anti-microbial activity of preservatives, so the amount of preservatives was increased in this formulation. In addition, Tween 80 may cause fat cell and subcutaneous adipose tissue thickenings after long term application; therefore Cream Formulation 10 should not be used longer than 3 months. This formulation also contains a high amount of glycerol, which serves to moisturize the skin and makes the cream more suitable to use in the treatment of psoriasis.

### Example 1.11

### Formulation 11

| Cream Base | | | |
|---|---|---|---|
| Stearic acid | 15.00 | g | (1) |
| White Vaselin | 10.00 | g | (2) |
| Monostearin | 8.50 | g | (3) |
| Propyl paraben | 0.28 | g | (4) |
| Methyl paraben | 0.11 | g | (5) |
| Vitamin A | 1.00 | g | (6) |
| Glycerol | 5.50 | | (7) |
| Aloe extracts in water | 2.00 | g | (8) |
| Tween 80 | 3.00 | g | (9) |
| De-ionized water | 51.11 | g | (10) |
| Primary Active Ingredient | | | |
| Meisoindigo | 0.50 | g | (11) |
| Other Ingredients | | | |
| Glycerol | 2.00 | g | (12) |
| Vitamin E | 1.00 | g | (13) |

### Procedures

Components (1) through (6) were melted by heating to 70° C. and mixed evenly. This mixture was filter-sterilized and maintained at 70° C. in a water bath until use (oil phase solution).
Components (7) and (9) were mixed evenly with de-ionized water (10), thereafter aloe extract (8) was added. This solution was filter-sterilized to obtain the final aqueous phase solution. The aqueous phase solution was maintained in a water bath at 70° C. until use.
Oil phase solution was then slowly added to aqueous phase solution with strong stirring, and an O/W cream base (Cream Base 11) was obtained and cooled to room temperature.
Suitable amount of Meisoindigo (11) was added to glycerol (12) and vitamin E (13) in a mortar and triturated deliberately. The O/W cream base was then added and continuously triturated to obtain Cream Formulation 11.
   Comments: Formulation 11 was therapeutically enhanced by adding Vitamins A and E, as well as aloe extract to moisturize the skin, which makes the cream more suitable to use in the treatment of psoriasis where itchiness and dryness are bothersome.

### Example 1.12

### Formulation 12

| Cream Base | | |
|---|---|---|
| Octadecanol | 9.00 g | (1) |
| White Vaselin | 10.00 g | (2) |
| Liquid paraffin | 6.00 g | (3) |
| Propyl paraben | 0.25 g | (4) |
| Methyl paraben | 0.10 g | (5) |
| Sodium lauryl alcohol sulfate | 1.00 g | (6) |
| Glycerol | 3.00 g | (7) |
| De-ionized water | 68.15 g | (8) |
| Primary Active Ingredient | | |
| Meisoindigo | 0.50 g | (9) |
| Other Ingredients | | |
| Dimethyl Sulfoxide (DMSO) | 1.00 g | (10) |
| Glycerol | 2.00 g | (11) |

### Procedures

Components (1) through (5) were melted by heating to 80° C. and mixed evenly. This mixture was filter-sterilized and maintained at 80° C. in a water bath until use (oil phase solution).
Sodium lauryl alcohol sulfate (6) was dissolved in de-ionized water (8) and then mixed evenly with glycerol (7). This solution was filter-sterilized to obtain the final aqueous phase solution. The aqueous phase solution was maintained in a water bath at 80° C. until use.
Oil phase solution was then slowly added to aqueous phase solution with strong stirring, and an O/W cream base (Cream Base 12) was obtained and cooled to room temperature.
Suitable amount of Meisoindigo (9) was dissolved with DMSO (10) in a mortar; thereafter glycerol (11) was added and triturated deliberately. The O/W cream base was then added and continuously triturated to obtain Cream Formulation 12.
   Comments: Since DMSO is an excellent solubilizer and skin penetration enhancer of meisoindigo, this formulation has improved therapeutic efficacy. In addition, Formulation 12 contains a high amount of moisturizer, so this preparation may be used in patients with moderate to severe psoriasis. However, DMSO may cause skin allergic reactions and is not suitable for patients with a history of skin allergies.

### Example 1.13

### Formulation 13

| Cream Base | | |
|---|---|---|
| Octadecanol | 9.00 g | (1) |
| White Vaselin | 10.00 g | (2) |
| Liquid paraffin | 6.00 g | (3) |
| Propyl paraben | 0.25 g | (4) |
| Methyl paraben | 0.10 g | (5) |
| Vitamin A | 1.00 g | (6) |
| Sodium lauryl alcohol sulfate | 1.00 g | (7) |
| Aloe extracts in water | 2.00 g | (8) |
| Glycerol | 5.00 g | (9) |
| De-ionized water | 63.65 g | (10) |
| Primary Active Ingredient | | |
| Meisoindigo | 0.50 g | (11) |
| Other Ingredients | | |
| DMSO | 0.50 g | (12) |
| Vitamin E | 1.00 g | (13) |

### Procedures

Components (1) through (6) were melted by heating to 80° C. and mixed evenly. This mixture was filter-sterilized and then maintained at 80° C. in a water bath until use (oil phase solution).
Sodium lauryl alcohol sulfate (7) was dissolved in de-ionized water (10) and then mixed evenly with glycerol (9) and aloe extracts (8). This solution was filter-sterilized to obtain the final aqueous phase solution. The aqueous phase solution was maintained in a water bath at 80° C. until use.
Oil phase solution was then slowly added to aqueous phase solution with strong stirring, and an O/W cream base (Cream Base 13) was obtained and cooled to room temperature.
Suitable amount of Meisoindigo (11) was dissolved with DMSO (12) in a mortar; thereafter vitamin E (13) was added and triturated deliberately. The O/W cream base was then added and continuously triturated to obtain Cream Formulation 13.
   Comments: Formulation 13 was enhanced by the addition of vitamins A and E, as well as aloe extracts. Furthermore the amount of DMSO was reduced in this formulation to minimize the risk of an allergic reaction.

### Example 1.14

### Formulation 14

| Cream Base | | |
|---|---|---|
| Methyl silicone oil | 20.00 g | (1) |
| Stearic acid | 15.00 g | (2) |
| Lanolin | 2.00 g | (3) |
| Vaselin | 7.00 g | (4) |
| Propyl paraben | 0.25 g | (5) |
| Methyl paraben | 0.10 g | (6) |
| Triethanolamine | 2.00 g | (7) |
| Glycerol | 2.00 g | (8) |
| De-ionized water | 48.65 g | (9) |
| Primary Active Ingredient | | |
| Meisoindigo | 0.50 g | (10) |
| Other Ingredients | | |
| DMSO | 0.50 g | (11) |
| Glycerol | 2.00 g | (12) |

### Procedures

Components (1) through (6) were melted by heating to 70° C. and mixed evenly. This mixture was filter-sterilized and maintained at 70° C. in a water bath until use (oil phase solution).
Triethanolamine (7) was dissolved in de-ionized water (9) and then mixed evenly with glycerol (8). This solution was filter-sterilized to obtain the final aqueous phase solution. The aqueous phase solution was maintained in a water bath at 70° C. until use.
Oil phase solution was then slowly added to aqueous phase with strong stirring, and an O/W cream base (Cream Base 14) was obtained and cooled to room temperature.
Suitable amount of Meisoindigo (10) was dissolved with DMSO (11) in a mortar and glycerol (12) was added and triturated deliberately. The O/W cream base was then added and continuously triturated to obtain Cream Formulation 14.
   Comments: Triethanolamine was used as emulsifer in this formulation. The presence of ions, such as Ca⁺⁺ and M⁺⁺, as well as acidic or alkalized conditions significantly decreases the stability of this formulation and should be avoided. Formulation 14 contains a high amount of oil, making it a moisture-locking cream suitable for dry skin.

### Example 1.15

### Formulation 15

| Cream Base | | |
|---|---|---|
| Methyl silicone oil | 20.00 g | (1) |
| Stearic acid | 15.00 g | (2) |
| Lanolin | 2.00 g | (3) |
| Vaselin | 5.00 g | (4) |
| Propyl paraben | 0.25 g | (5) |
| Methyl paraben | 0.10 g | (6) |
| Vitamin A | 1.00 g | (7) |
| Triethanolamine | 2.00 g | (8) |
| disodium EDTA | 0.04 g | (9) |
| Aloe extracts in water | 2.00 g | (10) |
| Glycerol | 5.00 g | (11) |
| De-ionized water | 45.86 g | (12) |
| Primary Active Ingredient | | |
| Meisoindigo | 0.50 g | (13) |
| Other Ingredients | | |
| DMSO | 0.25 g | (14) |
| Vitamin E | 1.00 g | (15) |

### Procedures

Components (1) through (7) were melted by heating to 70° C. and mixed evenly. This mixture was filter-sterilized and maintained at 70° C. in a water bath until use (oil phase solution).
Components (8) and (9) were dissolved in de-ionized water (13), pH was adjusted to 7.0 with either 0.01N HCl or 0.01N NaOH, and then mixed evenly with glycerol (11) and aloe extracts (10). This solution was filter-sterilized to obtain the final aqueous phase solution. The aqueous phase solution was maintained in a water bath at 70° C. until use.
Oil phase was then added to aqueous phase with strong stirring, and an O/W cream base (Cream Base 15) was obtained and cooled to room temperature.
Suitable amount of Meisoindigo (13) was dissolved with DMSO (14) and vitamin E (15) in a mortar and triturated deliberately. The O/W cream base was then added and continuously triturated to obtain Cream Formulation 15.
   Comments: EDTA was added to this formulation to improve stability. Formulation 15 was further enhanced by the addition of vitamins A and E, as well as aloe extracts. Furthermore the amount of DMSO was reduced in this formulation to minimize the risk of an allergic reaction.

### Example 1.16

### Formulation 16

| Cream Base | | |
|---|---|---|
| Vaseline ® Intensive Rescue | 99.00 g | (1) |
| Moisture Locking Lotion | | |
| Hypoallergenic Moisturizer | | |
| Primary Active Ingredient | | |
| Meisoindigo | 0.50 g | (2) |
| Other Ingredients | | |
| DMSO | 0.50 g | (3) |

### Procedures

Suitable amount of meisoindigo (2) was dissolved with DMSO (3) in a mortar and triturated deliberately. The O/W cream base (1) was then added and continuously triturated to obtain Cream Formulation 16.
   Comments: Vaseline Intensive Rescue Moisture Locking Lotion is a commercially available, hypoallergenic lotion suitable for dry skin. This cream contains water, glycerin, petrolatum, stearic acid, glycol stearate, dimethicone, isopropyl isostearate, tapioca starch, cetyl alcohol, glyceryl stearate, magnesium aluminum silicate, carbomer, ethylene brassylate, triethanolamine, disodium EDTA, phenoxyethanol, methyl paraben, propyl paraben, propyl paraben, and titanium dioxide. Thus, it was chosen as vehicle base to carry meisoindigo in Formulation 16. This formulation was used in Example 3.2.

### Example 1.17

### Formulation 17

| Liquid Base | | |
|---|---|---|
| Ethanol | 40.00 g | (1) |
| Glycerin | 7.00 g | (2) |
| De-ionized water | 52.94 g | (3) |
| Primary Active Ingredient | | |
| Meisoindigo | 0.01 g | (4) |
| Other Ingredients | | |
| DMSO | 0.05 g | (5) |

Comments: This formulation is designed for application as a topical spray. Thus, Formulation 17 is desirable when quick and easy application is an important factor to the patient. The patient need only hold the container roughly six to eight inches from the skin and spray the solution onto the affected epidermal area.

### Example 1.18

### (Formulation 18) Meisoindigo Gel

| Alcohol Solution | | |
|---|---|---|
| Glycerol | 7.00 g | (1) |
| Ethanol | 45.00 g | (2) |
| De-ionized water | 46.24 g | (3) |
| Primary Active Ingredient Solution | | |
| Meisoindigo | 0.010 g | (4) |
| DMSO | 0.25 g | (5) |
| Ethyl acetate | 0.50 g | (6) |
| Gelling Agent | | |
| Carbomer | 1.00 g | (7) |

### Procedures

Suitable amount of glycerol (1) and ethanol (2) were mixed with a suitable amount of de-ionized water (3) to obtain the alcohol solution.
Suitable amount of DMSO (5) and ethyl acetate (6) were added to a suitable amount of Meisoindigo (4) to obtain the primary active ingredient solution.
The primary active ingredient solution was then evenly mixed with the alcohol solution and filtered to obtain the mixed solution.
A suitable amount of carbomer was added evenly to the mixed solution to for a clear Meisoindigo gel.
   Comments: This gel formulation is cosmetically superior to Meisoindigo cream solutions in terms of appearance. It provides another easy way to use Meisoindigo for the topical treatment of psoriasis and other skin disorders. The addition of ethanol to this formulation increases skin penetration of the primary active ingredient (i.e. Meisoindigo), and less primary active ingredient is required. The concentration of the primary active ingredient, ethanol, and de-ionized water in this formulation may be adjusted, allowing for individual tailoring. For example, the concentration of Meisoindigo could be reduced to 0.005 g and the concentration of de-ionized water could concomitantly be increased to 46.245 g. In addition, the concentration of ethanol could be altered from Formula 18 by as much as 30%. The concentration of de-ionized water is adjusted with ethanol, while the other ingredients remain constant.

### Example 1.19

### (Formulation 19) Alcohol Spray

| Alcohol Solution | | |
|---|---|---|
| Glycerol | 7.00 g | (1) |
| Ethanol | 45.00 g | (2) |
| De-ionized water | 47.24 g | (3) |
| Primary Active Ingredient Solution | | |
| Meisoindigo | 0.010 g | (4) |
| DMSO | 0.25 g | (5) |
| Ethyl acetate | 0.50 g | (6) |

### Procedures

Suitable amount of glycerol (1) and ethanol (2) were mixed with a suitable amount of de-ionized water (3) to obtain the vehicle delivery solution.
Suitable amount of DMSO (5) and ethyl acetate (6) were added to a suitable amount of Meisoindigo (4) to obtain the primary active ingredient solution.
The primary active ingredient solution was then evenly mixed with the vehicle delivery solution and filtered to obtain the alcohol spray solution.
   Comments: This spray formulation provides a simple and effective way to use Meisoindigo for the topical treatment of psoriasis and other skin disorders. The addition of ethanol to this formulation increases skin penetration of the primary active ingredient (i.e. Meisoindigo), and less primary active ingredient is required. The concentration of the primary active ingredient, ethanol, and de-ionized water in this formulation may be adjusted, allowing for individual tailoring. For example, the concentration of Meisoindigo could be reduced to 0.005 g and the concentration of de-ionized water could concomitantly be increased to 47.245 g. In addition, the concentration of ethanol could be altered from Formula 18 by as much as 30%. The concentration of de-ionized water is adjusted with ethanol, while the other ingredients remain constant.

### Example 2

### Determination of Meisoindigo Stability in Creams by HPLC

An HPLC method was developed, validated, and used to determine concentrations and stability of meisoindigo in creams. The test method was validated with respect to precision, accuracy, range, and linearity as detailed below.

Meisoindigo cream was prepared in the same manner described in Example 1.2. An internal standard, N-ethylisoindigo, Lot#SNB-VII-193-3, was used in the HPLC analysis. N-ethylisoindigo was synthesized on Jan. 16, 2006, retested on Jun. 15, 2008, and expired on Jun. 15, 2009; its structure was confirmed by MNR and mass spectrometry.

An aliquot of cream was diluted and suspended in 9 volumes of water. The cream suspension was further diluted 10 times with methanol (HPLC Analytical Grade). This mixture was centrifuged at 3000 rpm for 10 min, and supernatant was used for HPLC analysis.

### Experimental Conditions

### HPLC

HPLC system: HP 1100
Detection: 270 nm
Column: Gemini C18, 250×4.6 mm, 5 μm
Column temperature: 25° C.
Flow rate: 1.0 mL/min
   Injection volume: 10 μL [injector program]
Mobile phase A: methanol
Mobile phase B: 0.05% H₃PO₄ in water
Sample diluent: Cream Base 2, de-ionized water and methanol
Run time: 25 minutes+5 minutes post time.

### Accuracy and Precision

Meisoindigo was added to 100 g of Cream Base 2 in the following amounts: 5 mg/mL (50%), 7.5 mg/mL (75%), 10 mg/mL (100%), 12.5 mg/mL (125%), and 15 mg/mL (150%). The samples were diluted by a factor of 10 in de-ionized water to obtain cream suspensions. These diluted cream suspensions were further diluted by a factor of 10 in methanol within a volumetric flask. After centrifugation at 3000 rpm for 10 minutes, 10p. 1 of the supernatant was injected into HPLC, and the peak area of meisoindigo was recorded (TABLE 1).

TABLE 1
Peak area of meisoindigo samples.

| Sample | 50% | 75% | 100% | 125% | 150% |
|---|---|---|---|---|---|
| Mean Area | 22445.12 | 26226.84 | 29111.94 | 29363.28 | 29550.08 |
| Std Dev | 154.453 | 41.857 | 67.082 | 47.017 | 68.872 |
| % RSD | 0.69 | 0.16 | 0.23 | 0.16 | 0.23 |

### Linearity

A series of dilutions was prepared from a stock solution (1.016 mg/mL) as outlined in TABLE 2, and HPLC quantification data are presented in TABLE 3.

TABLE 2
Dilution scheme for linear analysis.

| | | Final | |
|---|---|---|---|
| Dilution # | Pipette vol. (mL) | Dilution (mL) | Concentration (mg/mL) |
| Dil 1 | 5.0 (from stock) | 10.0 | 0.508 |
| Dil 2 | 5.0 (from Dil 1) | 10.0 | 0.254 |
| Dil 3 | 5.0 (from Dil 2) | 10.0 | 0.127 |
| Dil 4 | 5.0 (from Dil 3) | 10.0 | 0.064 |
| Dil 5 | 5.0 (from Dil 4) | 10.0 | 0.032 |
| Dil 6 | 5.0 (from Dil 5) | 10.0 | 0.016 |
| Dil 7 | 5.0 (from Dil 6) | 10.0 | 0.008 |

TABLE 3
HPLC quantification data of dilution series.

| Sample | Concentration (mg/mL) | Mean Area | Std Dev | % RSD |
|---|---|---|---|---|
| Stock | 1.016 | 28318.2 | 40.676 | 0.14 |
| Dil 1 | 0.508 | 21514.3 | 44.306 | 0.21 |
| Dil 2 | 0.254 | 12915.6 | 59.643 | 0.46 |
| Dil 3 | 0.127 | 6587.9 | 6.591 | 0.10 |
| Dil 4 | 0.064 | 3312.1 | 4.440 | 0.13 |
| Dil 5 | 0.032 | 1640.6 | 3.340 | 0.20 |
| Dil 6 | 0.016 | 827.2 | 1.530 | 0.19 |
| Dil 7 | 0.008 | 411.2 | 0.490 | 0.12 |

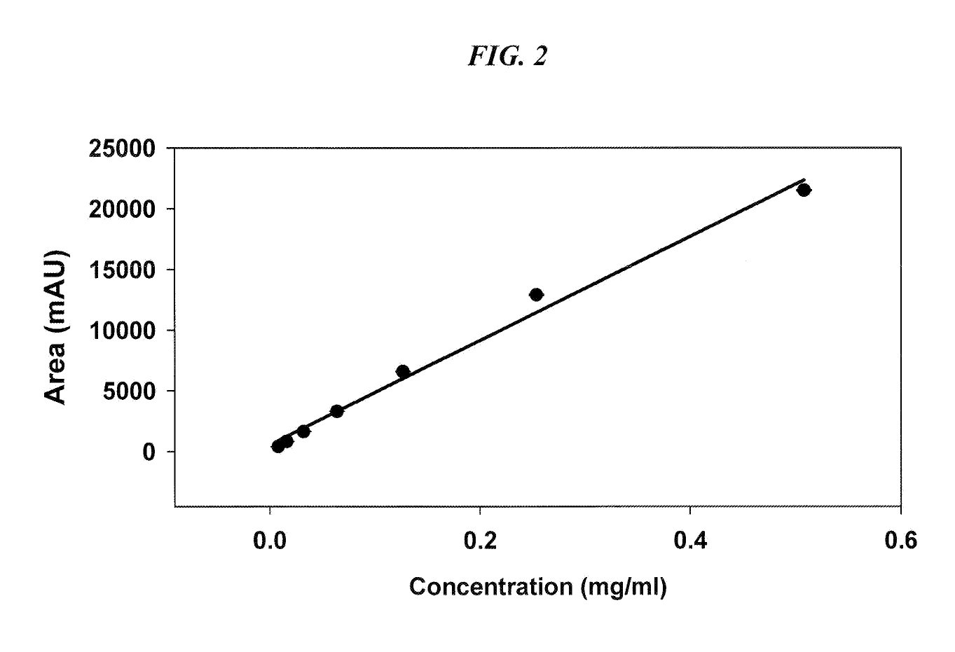

Linearity was examined in a range from 0.508 mg/mL to 0.008 mg/mL, and the correlation coefficient was found to be 0.9999. A graphical presentation of the linear response is presented in FIG. 2.

### Limits of Detection and Quantitation

A stock meisoindigo standard was prepared (10.01 mg/g of Cream Base 2) and diluted by a factor of 10 with de-ionized water. Thereafter, a series of dilutions were prepared in methanol (TABLE 4). Results indicated that the detection limit of meisoindigo is 0.01 mg/mL.

TABLE 4
Dilution scheme and HPLC detection data.

| | | Final | | | |
|---|---|---|---|---|---|
| | Pipette vol. | Dilution | Concentration | Mean | % |
| Sample | (mL) | (mL) | (mg/mL) | Area | RSD |
| Stock | — | — | 1.0012 | — | — |
| Dil 1 | 1.0 (from stock) | 10.0 | 0.10012 | 22139.76 | 0.69 |
| Dil 2 | 1.0 (from Dil 1) | 10.0 | 0.010012 | 586.52 | 0.24 |
| Dil 3 | 1.0 (from Dil 2) | 10.0 | 0.0010012 | 64.641 | 0.38 |
| Dil 4 | 1.0 (from Dil 3) | 10.0 | 0.00010012 | 11.79 | 8.12 |
| Dil 5 | 1.0 (from Dil 4) | 10.0 | 0.000010012 | 5.01 | 25.34 |

### Standard Stability

The six-month stability of Cream Formulation 2 when stored at room temperature was assayed, and the data are presented in TABLE 5. As a preliminary observation, the cream exhibited no change in appearance. These results indicate that Cream Formulation 2 is stable for at least six months when stored at room temperature.

TABLE 5
Percentage of meisoindigo detected in sample at various storage times.

| Sample Time | Meisoindigo Concentration (%) |
|---|---|
| Initial | 100.39 |
| 1 month | 100.28 |
| 3 Months | 100.84 |
| 6 Months | 100.71 |

### Example 3

### Efficacy and Side Effects of Skin Cream in Treating Psoriasis

### Example 3.1

The efficacy of Cream Formulation 2 was studied on a 53 year-old male patient suffering from psoriasis for approximately 25 years. The patient exhibited psoriatic lesions primarily on the skin around the ankles, knees and elbows. The psoriatic skin lesions were characterized by erythematic patches, which were itchy and dry with a whitish, scaly appearance. The patient reported periods of exacerbation lasting 3-4 months when the patches grow larger, extending from the knee to the calf and thigh and from the elbows to the upper and lower arms. In addition, itching becomes unbearable and massive shedding occurs, leaving scales in the bed and clothing. The underlying skin turns bright red and friable with spontaneous bleeding upon scrubbing during bathing. Furthermore, the patient was hesitant to wear clothing that did not cover these lesions when out in public.

Within the month preceding application of Cream Formulation 2, the patient suffered from exacerbation of psoriatic dermatitis with extension of the lesions to both the calf and thigh area associated with severe itching, desquamation, and spontaneous bleeding of the affected areas. This episode was particularly severe, and the patient was disturbed by the failure of the usual ointments prescribed by his dermatologist to palliate the symptoms. Therefore, the dermatologist proposed Remicade (infliximab), but the patient was refused, fearful of the possible side effects (e.g., serious infections caused by viruses, fungi, or bacteria, including tuberculosis, histoplasmosis, and pneumonitis).

The patient began treatment with Cream Formulation 2, applying the cream on dry skin twice daily (morning and night). Improvement in the affected areas was noticeable within the first seven to ten days of treatment. The desquamation, redness, and itching resolved completely in the calf and thighs. However, some scaly areas persisted, but attenuated, around the ankles. Approximately one month later, the patient discontinued treatment due to significant palliation of symptoms and regeneration of the skin in the affected areas. Significantly, the patient reported no side effects associated with treatment. Furthermore, the patient has not suffered another episode of exacerbation since the period of treatment (Aug. 23-Sep. 20, 2008), and thus has not used Cream Formulation 2 again.

### Example 3.2

### Study Design

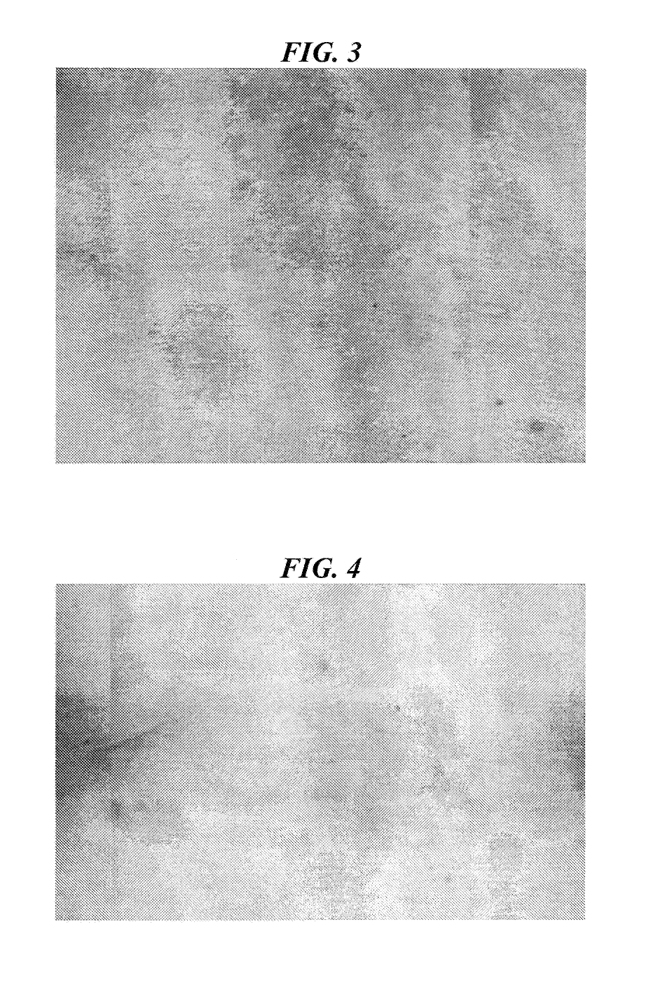

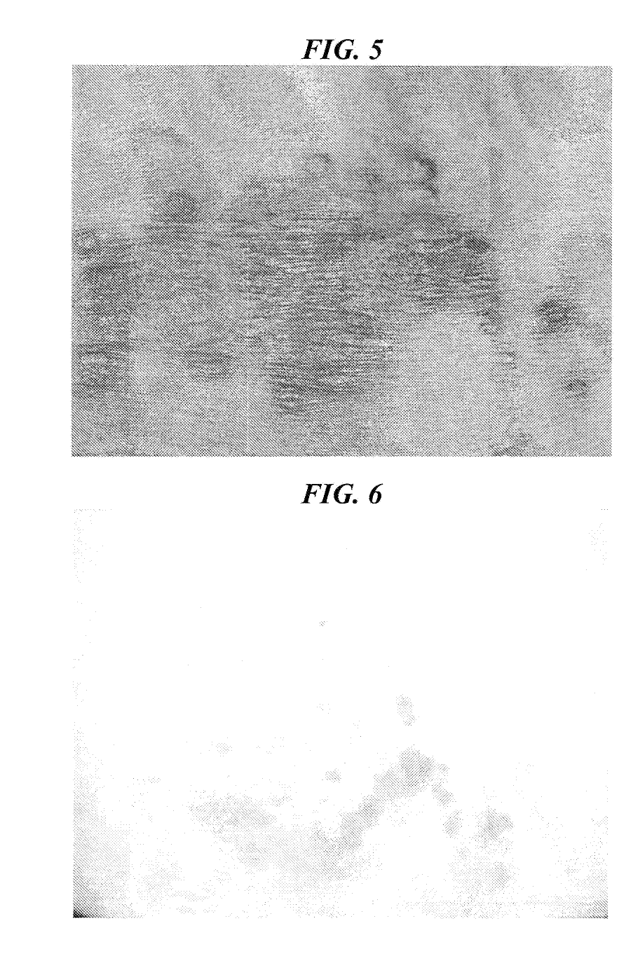

A total of 10 healthy men (n=5) and women (n=5) were enrolled in the study. The average age of the subjects was 51 (ranging from 27 to 71 years). The racial backgrounds of the subjects included Caucasian (n=6), Hispanic/Latino (n=3), and African American (n=1). All subjects had moderate to severe plaque psoriasis covering at least 5 cm² of their body. The location of the psoriasis was easily accessible for treatment and photographing. After each subject reviewed and signed a consent form approved by the Essex Institutional Review Board (Lebanon, N.J.), eligibility screening was performed and medical history recorded. The Principal Investigator (PI) rated the severity of the plaque psoriasis to insure each subject's eligibility for participation. At the eligibility visit, each subject was given instructions for use of DermoKare (Formulation 16) and a diary to track twice-daily applications and any unexpected outcomes. Instructions included directions to cleanse the affected area prior to application, and each subject applied the first application under the observation of the study coordinator. Each eligible subject was photographed (see FIGS. 3 and 5) and then given enough DermoKare to last for 28 days (±3 days) with twice-a-day use. Each subject was seen for a total of two or four follow-up visits: 14 days (±3 days), 28 days (±3 days), 42 days (±3 days), and 56 days (±3 days). At the 28-day visit each subject was assessed for overall improvement according to the Psoriasis Area and Severity Index (PASI). If the PI determined that reduction in a subject's PASI score was greater than 50% (51% to 100%), the subject's enrollment in the study was concluded and all end-of-study paperwork was completed. An improvement of greater than 50% was deemed an appropriate end point because such a reduction in PASI score demonstrates a clinically meaningful benefit (Carlin et al. 2004). If a patient's response to treatment was 50% or less (0% to 50%), his or her enrollment continued for an additional 4 weeks. Subjects who continued with treatment were given enough additional DermoKare to last the remainder of the study. At each follow-up visit the subject was photographed (see FIGS. 4 and 6) and improvement assessed. A summary of the study protocols is given in TABLE 6.

TABLE 6
DermoKare Study Protocols.

| | Eligibility | Visit 1 | Visit 2 | Visit 3 | Visit 4 |
|---|---|---|---|---|---|
| Activity | Visit | (d 14) | (d 28) | (d 42) | (d 56) * |
| Informed Consent | X | | | | |
| Eligibility/Medical | X | | | | |
| History | | | | | |
| PI Assessment of | X | | | | X |
| Psoriasis | | | | | |
| Subject Assessment of | X | | | | X |
| Psoriasis | | | | | |
| PI Assessment of | X | | | | X |
| Erythema, Scaling, | | | | | |
| and Pruritis | | | | | |
| Photographs | X | X | X | X | X |
| PI Assessment of | | X | X | X | X |
| Improvement in | | | | | |
| Psoriasis | | | | | |
| Subject Assessment of | | X | X | X | X |
| Improvement in | | | | | |
| Psoriasis | | | | | |
| PI Assessment of | | X | X | X | X |
| Improvement in | | | | | |
| Erythema, Scaling | | | | | |
| & Pruritis | | | | | |
| Distribution of | X | | X | | |
| DermoKare | | | | | |
| Subject Satisfaction | | | | | X |
| Questionnaire | | | | | |
| Concomitant | X | X | X | X | X |
| Medications | | | | | |
| Adverse Events | | X | X | X | X |
| End of Study/ | | | | | X |
| Termination | | | | | |

* Visits 3 and 4 occurred only if the subject exhibited 50% or less improvement in his or her psoriasis as measured by PASI. If the study concluded on Visit 2 for a particular subject, end of study procedures were performed at that time.

### Statistical Methods

A paired, two-tailed student's t-test was used to analyze significances before and after treatment. Descriptive statistics (number of observations, arithmetic mean, standard deviation, median, minimum and maximum) were calculated using SAS, Version 8.2 (SAS Institute, Cary, N.C., USA) for quantitative efficacy and safety data. Adverse events were coded using the MedDRA dictionary (Version 12.0) and summarized. Prior and concomitant medications were coded using the World Health Organization (WHO) Drug Dictionary Version 9.0 and listed by reported term and Anatomical Therapeutic Class (ATC) classification.

### Results—Efficacy

Of the ten subjects participating in the study, nine exhibited greater than 50% improvement as measured by the PASI within 4 weeks. Therefore only one subject remained in the study for the full eight weeks (TABLE 7). The following criteria were used to evaluate efficacy: quartile scale: 0-25%, 26-50%, 51-75%, 76-100%); severity scale (itching, redness, and scaling; 0=None, 1=Some, 2=Moderate, 3=Severe, 4=Maximum); and body coverage score 0=0%; 1=<10%; 2=10-29%, 3=30-49%, 4=50-69%, 5=70-89%, 6=80-100%). These results are listed in TABLES 8-11. Exemplary photographs of affected epidermal areas of two patients participating in the study are provided in FIGS. 3-6.

TABLE 7
Summary of DermoKare on Overall Improvement (PASI).

| Patient # | Weeks of Treatment | PI Assessment | Subject Assessment |
|---|---|---|---|
| 1 | 4 | 76-100% | 76-100% |
| 2 | 4 | 76-100% | 51-75% |
| 3 | 4 | 51-75% | 51-75% |
| 4 | 4 | 51-75% | 26-50% |
| 5 | 4 | 76-100% | 51-75% |
| 6 | 8 | 51-75% | 51-75% |
| 7 | 4 | 76-100% | 0-25% |
| 8 | 4 | 51-75% | 26-50% |
| 9 | 4 | 76-100% | 76-100% |
| 10 | 4 | 76-100% | 76-100% |

TABLE 8
Summary of DermoKare treatment on the Severity of Itching.*

| | PI Assessment | | Subject Assessment | |
|---|---|---|---|---|
| Patient # | Eligibility Visit | End of Study | Eligibility Visit | End of Study |
| 1 | 4 | 0 | 4 | 1 |
| 2 | 1 | 0 | 1 | 0 |
| 3 | 2 | 0 | 0 | 0 |
| 4 | 3 | 0 | 2 | 1 |
| 5 | 3 | 0 | 3 | 0 |
| 6 | 3 | 0 | 2 | 0 |
| 7 | 3 | 0 | 2 | 2 |
| 8 | 2 | 1 | 1 | 2 |
| 9 | 3 | 0 | 3 | 0 |
| 10 | 0 | 0 | 0 | 0 |
| Mean | 2.4 | 0.1 | 1.8 | 0.6 |
| Std Dev. | 1.2 | 0.3 | 1.3 | 0.8 |

| | | |
|---|---|---|
| p value | p < 0.0003 | p = 0.0301 |

*Severity scale: 0 = None, 1 = Some, 2 = Moderate, 3 = Severe, 4 = Maximum

TABLE 9
Summary of DermoKare treatment on the Severity of Redness.*

| | PI Assessment | | Subject Assessment | |
|---|---|---|---|---|
| Patient # | Eligibility Visit | End of Study | Eligibility Visit | End of Study |
| 1 | 2 | 0 | 3 | 1 |
| 2 | 3 | 1 | 3 | 1 |
| 3 | 0 | 0 | 0 | 1 |
| 4 | 4 | 1 | 3 | 1 |
| 5 | 4 | 1 | 1 | 1 |
| 6 | 4 | 2 | 2 | 1 |
| 7 | 2 | 1 | 3 | 2 |
| 8 | 2 | 1 | 2 | 2 |
| 9 | 1 | 0 | 1 | 1 |
| 10 | 2 | 1 | 2 | 1 |
| Mean | 2.4 | 0.8 | 2.0 | 1.2 |
| Std Dev. | 1.3 | 0.6 | 1.1 | 0.4 |

| | | |
|---|---|---|
| p value | p = 0.0024 | p = 0.0444 |

*Severity scale: 0 = None, 1 = Some, 2 = Moderate, 3 = Severe, 4 = Maximum

TABLE 10
Summary of DermoKare treatment on the Severity of Scaling.*

| | PI Assessment | | Subject Assessment | |
|---|---|---|---|---|
| Patient # | Eligibility Visit | End of Study | Eligibility Visit | End of Study |
| 1 | 3 | 0 | 4 | 0 |
| 2 | 3 | 0 | 3 | 0 |
| 3 | 3 | 0 | 2 | 0 |
| 4 | 3 | 1 | 3 | 1 |
| 5 | 4 | 0 | 2 | 0 |
| 6 | 4 | 2 | 2 | 2 |
| 7 | 3 | 1 | 3 | 2 |
| 8 | 4 | 1 | 2 | 1 |
| 9 | 2 | 1 | 2 | 0 |
| 10 | 3 | 1 | 3 | 1 |
| Mean | 3.2 | 0.7 | 2.6 | 0.7 |
| Std Dev. | 0.6 | 0.7 | 0.7 | 0.8 |

| | | |
|---|---|---|
| p value | p < 0.0001 | p < 0.0001 |

*Severity scale: 0 = None, 1 = Some, 2 = Moderate, 3 = Severe, 4 = Maximum

TABLE 11
Summary of DermoKare treatment on the Percentage of Body Area
Covered by Psoriasis.*

| | PI Assessment | | Subject Assessment | |
|---|---|---|---|---|
| Patient # | Eligibility Visit | End of Study | Eligibility Visit | End of Study |
| 1 | 2 | 0 | 2 | 0 |
| 2 | 4 | 1 | 3 | 1 |
| 3 | 3 | 3 | 2 | 1 |
| 4 | 5 | 2 | 3 | 1 |
| 5 | 4 | 2 | 2 | 1 |
| 6 | 6 | 5 | 3 | 3 |
| 7 | 1 | 1 | 1 | 1 |
| 8 | 2 | 1 | 1 | 1 |
| 9 | 1 | 1 | 1 | 1 |
| 10 | 1 | 1 | 2 | 1 |
| Mean | 2.9 | 1.7 | 2.0 | 1.1 |
| Std Dev. | 1.8 | 1.4 | 0.8 | 0.7 |

| | | |
|---|---|---|
| p value | p = 0.0130 | p = 0.0100 |

*Coverage scale: 0 = 0%, 1 = <10%, 2 = 10-29%, 3 = 30-49%, 4 = 50-69%, 5 = 70-89%, 6 = >89%

Subjects were also asked to complete a satisfaction questionnaire at the conclusion of the study. Specifically, the following question was posed: How do you feel about the appearance of your treatment area? Subjects could choose the following responses:

I am very dissatisfied with the appearance of my treatment area

I am dissatisfied with the appearance of my treatment area

I am somewhat satisfied with the appearance of my treatment area

I am satisfied with the appearance of my treatment area

I am very satisfied with the appearance of my treatment area

Two subjects were somewhat satisfied, four were satisfied, and four were very satisfied. Thus, none of the subjects in the study were dissatisfied with their results post-treatment.

### Results—Adverse Events

Definitions and instructions for monitoring, recording and reporting adverse events were reviewed with investigational site personnel prior to enrollment. An adverse event was defined as any untoward medical occurrence in a patient, which does not necessarily have to have a causal relationship with treatment. An adverse event can therefore be any unfavorable and unintended sign (including an abnormal laboratory finding assessed as clinically significant and different from the baseline), symptom, or disease temporally associated with the use of the investigational product, whether or not related to the investigational product. Thus, any new sign, symptom or disease, or clinically significant increase in the intensity of an existing sign, symptom or disease, was considered as an adverse event. A serious adverse event is any untoward medical occurrence that results in death, is life threatening, requires inpatient hospitalization or prolongation of existing hospitalization, results in persistent or significant disability/incapacity, or is a congenital anomaly/birth defect. Significantly, no adverse event was reported throughout this clinical trial.

### Discussion

Both studies in Example 3 suggest that topical meisoindigo effectively treats psoriasis. There are currently no biomarkers available to assess the severity of psoriasis. Thus, PASI is the most accepted and widely used measure in clinical trials (Carlin et al. 2004). However, PASI suffers from significant limitations including subjectivity, lack of consistency between evaluators, variation of primary endpoint, and the fact the improvement is experienced differently by different patients. Nonetheless, more information may be gleaned by comparing the results in Example 3.2 with those reported in studies evaluating the efficacy of oral meisoindigo (TABLE 12).

TABLE 12
Comparison of DermoKare application vs. oral administration.

| Study | Example 3.2 | | Lin et al. (1989) | | Unpublished (1993)¹ | |
|---|---|---|---|---|---|---|
| No. of | 10 | | 40 | | 103 | |
| Patients | | | | | | |
| Dosage | lotion 0.5% | | 75 or 150 mg | | 100 mg for 2 weeks | |
| | | | | | followed by 150 mg for 4 | |
| | | | | | weeks | |
| Duration | 4 weeks, n = 9 | | 8 weeks average | | 6 weeks | |
| of | 8 weeks, n = 1 | | Min = 1 week | | | |
| treatment | | | Max = 22 weeks | | | |
| | PASI | % of | PASI | % of | PASI | % of |
| Efficacy | (% improved) | patients | (% improved) | patients | (% improved) | patients |
| | 76-100 | 60 | remission^{2,3} | 30 | remission² | 11 |
| | 51-75 | 40 | 50-90 | 30 | >70 | 22 |
| | ≦50 | 0 | <50 | 20 | 30-70 | 42 |
| | 0 | 0 | 0 | 20 | <30 | 25 |

| | % of patients reporting | % of patients reporting | | % of patients reporting | |
|---|---|---|---|---|---|
| | side effects | side effects | | side effects | |
| Side | No side effects | facial | 17.5 | GI | 25.2 |
| effects | reported | | swelling | | symptoms |
| | | abdominal | 7.5 | muscle pain | 3.9 |
| | | pain | | | |
| | | nausea | 2.5 | | |
| | | diarrhea | 2.5 | | |

¹Compiled data from four hospitals in China; data available upon request.
²Grade defined by the Chinese Ministry of Health; complete clearance of damaged skin, reduction of erythema, and disappearance of itching, scaling, and burning.
³For patients reaching the level of remission, the average duration of treatment was 10 weeks.

Treatment of psoriasis with DermoKare improved PASI by at least 51% in all patients, and by at least 76% in 60% of patients. Contrastingly, as reported by Lin et al. (1989), treatment of psoriasis with oral meisoindigo achieved at least 50% improvement in PASI in 60% of patients; and, as reported in the 1993 study, oral meisoindigo achieved greater than 70% improvement in PASI in 33% of patients. Thus, it appears that DermoKare is a more effective psoriasis treatment than oral meisoindigo. In fact, the data suggest that DermoKare is nearly twice as effective as oral meisoindigo. Moreover, treatment with oral meisoindigo caused a variety of side effects, most commonly gastrointestinal problems and facial swelling.

DermoKare may also be compared to other common therapies used to treat psoriasis. Aloria-Palli et al. (2010) studied the effects of a twelve-week treatment with topical liquor carbonis distillate (coal tar) or calcipotriol on sixty patients suffering from moderate plaque psoriasis. The authors reported treatment with coal tar achieved an average improvement in PASI of 58% and treatment with calcipotriol achieved an average improvement in PASI of 37%. Patients receiving coal tar reported exacerbation of psoriasis (n=2), folliculitis (n=2), and a phototoxic reaction (n=1), and patients receiving calcipotriol reported exacerbation of psoriasis (n=2), an irritant contact dermatitis (n=1), and an application site reaction (n=1). Kragballe et al. (2004) studied the effects of (1) treatment with a compound comprising calcipotriol and betamethasone dipropionate (BD) for eight weeks followed by calcipotriol alone for four weeks; (2) treatment with calcipotriol/BD for four weeks followed by calcipotriol alone on weekdays and calcipotriol/BD on weekends for eight weeks; and (3) treatment with calcipotriol alone for twelve weeks. The study involved 972 patients with mild, moderate, or severe forms of psoriasis. At the end of eight weeks of treatment, the authors reported improvements in PASI of 73.3%, 68.2%, and 64.1% for groups 1, 2, and 3, respectively. In addition, adverse drug reactions were recorded for 10.9%, 11.5%, and 22.3% of patients receiving treatments 1, 2, and 3, respectively. Griffiths et al. (2010) studied the effects of treatment with ustekinumab or etanercept on 903 patients with moderate-to-severe psoriasis. Ustekinumab was injected at weeks 0 and 4 while patients on the etanercept treatment received twice weekly doses. The authors reported that 71.4% of those patients receiving ustekinumab, and 56.8% of those patients receiving etanercept, achieved at least 75% improvement in PASI score within twelve weeks. Adverse events were recorded in 68.0% (1.2% classified as serious) of those patients receiving ustekinumab and 70.0% (1.4% classified as serious) of those patients receiving etanercept.

When compared to other common psoriasis treatments, DermoKare performed advantageously. The efficacy of DermoKare appears to be similar to that of potent psoriasis medications. However, no side effects have been reported with DermoKare treatment. Admittedly, the sample size is very small, but DermoKare is not likely to cause side effects to the degree reported with calcipotriol, betamethasone dipropionate, ustekinumab, or etanercept.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A method of treating a psoriasis, the method comprising the step of topically applying a composition comprising meisoindigo in a pharmaceutically acceptable carrier to the affected epidermal area on a subject in need.

2. The method of claim 1, wherein meisoindigo is between 0.001 to 5.00 percent by weight of the composition.

3. The method of claim 2, wherein meisoindigo is N-methyl-Δ3,3′-dihydroindole-2,2′ diketone.

4. The method of claim 3, wherein the composition is topically administered twice a day.

5. The method of claim 3, wherein the composition is administered every day for no longer than six consecutive months.

6. The method of claim 3, wherein the composition further comprises an additional active agent.

7. The method of claim 6, wherein the additional active agent is selected from the group consisting of alefacept, efalizumab, infliximab, adalimumab, etanercept, ustekinumab, methotrexate, cyclosporine, and phototherapy.

8. The method of claim 2, wherein the subject is a human.

9. A topical composition for treating psoriasis comprising a therapeutically effective amount of meisoindigo and a pharmaceutically acceptable topical carrier.

10. The topical composition of claim 9, wherein meisoindigo is N-methyl-Δ3,3′-dihydroindole-2,2′ diketone in an amount between 0.001 to 5.00% by weight of the topical composition.

11. The topical composition of claim 10, wherein the composition is an emulsion comprising an oil phase component, an aqueous phase component, an emulsifier, a moisturizer, a skin penetration enhancer, and a surfactant or solubility enhancer.

12. The topical composition of claim 11, comprising the following ingredients by weight:
the oil phase component at 1.00 to 85.00%;
the aqueous phase component at 1.00 to 85.00%;
the emulsifier at 0.01 to 10.00%;
the moisturizer at 0.50 to 25.00%;
the skin penetration enhancer at 0.01 to 10.00%; and
the surfactant or solubility enhancer at 0.01 to 10.00%.

13. The topical composition of claim 12, wherein:
the oil phase component is selected from the group consisting of glyceryl monoacetate, glycerol diacetate, glyceryl triacetate, stearic acid, soybean oil, corn oil, peanut oil, palmitic acid, palm oil, sunflower oil, olive oil, coconut oil, sesame oil, cotton seed oil, low erucic acid rapeseed oil, oleic acid, medium-chain triglycerides, single-decane triglyceride, lanolin, beeswax, petrolatum, hydrocarbons, and Vaseline®;
the aqueous phase component is selected from the group consisting of de-ionized water, glycerol gelatin, microcrystalline cellulose, PEG 300, PEG 400, PEG 4000, and PEG 6000;
the emulsifier is selected from the group consisting of magnesium stearate, milk amino acids, sodium lauryl sulfate, triethanolamine, magnesium aluminum silicate, and a carbomer;
the moisturizer is selected from the group consisting of glycerol, propylene glycol, and sorbitol;
the skin penetration enhancer is selected from the group consisting of dimethyl sulfoxide, laurocapram, 2-pyrrolidone), ethanol, decanol, oleic acid, propylene glycol, dimethylformamide, dimethylacetamide, lauryl alcohol, fatty acid esters, fatty acids, EO-2-oleyl ether, terpenes, and lecithin; and
the surfactant or solubility enhancer is selected from the group consisting of dimethyl sulfoxide, K17, K25, K30, K90), PEG 400, PEG 4000, PEG 6000, stearic acid hydrocarbon oxygen Poly (40) ester, lauryl alcohol polyoxyethylene (23) ether, vitamin E succinate polyethylene glycol ester, polyoxyethylene (40) hydrogenated castor oil, and polyoxy (40) stearate.

14. The topical composition of claim 11, wherein the solubility enhancer is ethyl acetate.

15. The topical composition of claim 12, wherein the aqueous phase component is de-ionized water, the emulsifier is carbomer, the moisturizer is glycerol, and the skin penetration enhancer is ethanol.

16. The topical composition of claim 11 further comprising at least one of: an antioxidant, vitamin, lubricant, herbal extract, preservative, or other ingredients.

17. The topical composition of claim 16 comprising at least one of the following ingredients by weight:
an antioxidant at 0.01 to 10.00%;
a vitamin at 0.01 to 10.00%;
a lubricant at 0.01 to 10.00%;
an herbal extract at 0.01 to 10.00%;
a preservative at 0.01 to 10.00% by weight of the composition; and
an other ingredient at 0.01 to 20.00%.

18. The topical composition of claim 15, wherein:
the antioxidant is selected from the group consisting of vitamin C, vitamin C palmitate, propyl gallate, vitamin E, and tert-butyl ether-hydroxybenzoate fennel;
the vitamin is selected from the group consisting of vitamin A, vitamin B series, vitamin C, vitamin D, and vitamin E;
the lubricant is selected from the group consisting of magnesium stearate, sodium lauryl sulfate, polyethylene glycol, and silica gel powder;
the herbal extract is aloe vera;
the preservative is selected from the group consisting of hydroxylethyl benzene, hydroxylmethyl benzene, phenoxyethanol, propyl paraben, and methyl paraben; and
the other ingredient is selected from the group consisting of keratin, collagen, amino acids, lecithin, aloe extracts, dimethicone, and disodium EDTA.

19. The topical composition of claim 10, wherein the composition is an alcohol-based spray comprising de-ionized water, ethanol, at least one moisturizer, at least one skin penetration enhancer, and at least one surfactant or solubility enhancer.

20. The topical composition of claim 19, wherein the moisturizer is glycerol and the skin penetration enhancer is dimethyl sulfoxide.

21. The topical composition of claim 21, wherein the solubility enhancer is ethyl acetate.

22. The topical composition of claim 10, further comprising a second active agent.

23. The topical composition of claim 22, wherein the second active agent is selected from the group consisting of: coal tar, calcipotriol, corticosteroids, retinoids, vitamin D, vitamin E, aloe extracts, herbal remedies, analgesics, and nonsteroidal anti-inflammatory drugs.

24. A topical composition comprising the following ingredients by weight:
meisoindigo at between 0.001 to 5.00%;
dimethyl sulfoxide at between 0.10 to 5.00%; and
petrolatum at between 1.00 to 70.00%.
